(19) United States
(12) Patent Application Publication
(10) Pub. No.: US 2024/0426811 A1
(43) Pub. Date: Dec. 26, 2024

(54) SCREENING FOR PHARMACEUTICAL COMPOUNDS FOR IMPROVING MITOCHONDRIAL FUNCTION

(71) Applicant: Centaurus Therapeutics, Half Moon Bay, CA (US)

(72) Inventor: Jeremy Blitzer, San Francisco, CA (US)

(21) Appl. No.: 18/609,816

(22) Filed: Mar. 19, 2024

Related U.S. Application Data
(60) Provisional application No. 63/453,406, filed on Mar. 20, 2023.

(51) Int. Cl. G01N 33/50

(52) U.S. Cl. CPC G01N 33/5079

(57) ABSTRACT

The present disclosure is directed to methods of screening candidate agents for improving mitochondrial function in a subject and assaying kits for use thereof. The present disclosure further provides methods of treating mitochondrial dysfunction in a subject in need of treatment thereof by administering such compounds identified by the methods.

### CROSS-REFERENCE TO RELATED APPLICATIONS

This application claims the benefit of priority to U.S. Provisional Application Ser. No. 63/453,406, filed Mar. 20, 2023, the contents which are hereby incorporated by reference in their entirety.

### TECHNICAL FIELD

The present disclosure generally relates to performing screening assays, more specifically, methods of screening candidate agents for improving mitochondrial function in a subject and assaying kits for use thereof. The present disclosure further provides methods of treating mitochondrial dysfunction in a subject in need of treatment thereof by administering such compounds identified by the methods.

### BACKGROUND

Mitochondria are the energy factory of our body. Several thousand mitochondria are in nearly every cell in the body. The functions of mitochondria include oxidative phosphorylation, which generates ATP by utilizing the energy released during the oxidation of the food we eat. More mitochondria are needed to make more energy, particularly in high-energy demand organs such as the heart, muscles, kidney, liver, and brain. When the number or function of mitochondria in the cell are disrupted, less energy is produced and organ dysfunction results. As such, mitochondrial dysfunction refers to a heterogeneous group of disorders resulting in defective cellular energy production due to abnormal oxidative phosphorylation. Mitochondrial dysfunction also can cause a large variety of human disorders, such as cardiomyopathy, neurodegeneration, rhabdomyolysis, cancer, migraine, obesity, diabetes, infertility, kidney, and liver diseases and many more.

However, there has been no effective treatment or cure for mitochondrial dysfunction. Treatment for mitochondrial dysfunction varies from patient to patient and depends on the specific mitochondrial disease diagnosed and its severity. Thus, there remains a need to find pharmaceutical compounds as potential new therapeutic drugs for treating mitochondria dysfunction.

### SUMMARY OF THE INVENTION

Provided are methods of screening for a pharmaceutical compound for improving mitochondrial function. Specifically, the methods comprise a) contacting a biological sample obtained from a subject with a candidate agent; and b) measuring mitochondrial function in the sample, wherein the presence of an elevated level of mitochondrial function compared to that in a negative control that is not treated with the candidate agent indicates that the candidate agent improves mitochondrial function. Also provided are methods of treating a subject in need of treatment thereof for mitochondrial dysfunction by administering such compounds identified by the methods, as described herein. Further provided are assaying kits for use thereof.

In one aspect, the biological sample obtained from a subject is an impaired tissue associated with mitochondrial dysfunction. For example, the biological sample is muscle tissue, brain tissue, liver tissue, heart tissue, kidney tissue, pancreas, adipose tissue, gastrointestinal tract tissue or blood plasma. In some embodiments the biological sample is H9C2 rat cardiomyoblast cells or cultured cardiomyocytes from a human with a fatty acid oxidation disorder. The subject suffers from mitochondrial dysfunction or disease associated with mitochondrial dysfunction. In some instances, mitochondrial dysfunction or the disease associated with mitochondrial dysfunction includes, but is not limited to, metabolic disease, skeletal muscle disease, cardiac disease, liver disease, diabetes, obesity, kidney disease, pancreas disorder, cancer, or fatty acid oxidation disorders (FAODs).

In another aspect, the candidate agent for improving mitochondrial function may be a small molecule, a peptide, a protein, enzyme, an antibody, an antibody mimetic, an aptamer, or an inhibitory nucleic acid. In certain instances, the candidate agent is a dihydroceramide desaturase (Des) inhibitor.

In still another aspect, the mitochondrial function in the sample is measured by oxygen consumption rate (OCR) and/or extracellular acidification rate (ECAR).

### DETAILED DESCRIPTION

The present disclosure provides methods for screening pharmaceutical compounds for improving mitochondrial function. The present disclosure further provides methods of treating mitochondrial dysfunction in a subject in need of treatment thereof by administering such compounds identified by the subject methods. In some embodiments, methods of interest may include determining efficacy of candidate agents in improving mitochondrial dysfunction or treating mitochondrial dysfunction. Kits for screening pharmaceutical compounds for improving mitochondrial function are also provided.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### I. Definitions

As used herein, the terms below have the meanings indicated.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

When introducing elements of the present disclosure or the preferred embodiment(s) thereof, the articles “a”, “an”, “the” and “said” are intended to mean that there are one or more of the elements. Thus, for example, reference to “an enzyme” includes a plurality of such enzymes and reference to “the inhibitor” includes reference to one or more inhibitors and equivalents thereof, e.g., antagonists, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, the term “about,” particularly in reference to a given quantity, is meant to encompass deviations of plus or minus five percent.

As used herein, the term “administering” is intended to include routes of administration which allow the agent to perform its intended function of improving mitochondrial activity/function in a subject. Examples of routes of administration which can be used include injection (subcutaneous, intravenous, parenterally, intraperitoneally, intrathecal, etc.), oral, inhalation, and transdermal. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. Further, the agent may be coadministered with a pharmaceutically acceptable carrier. The agent also may be administered as a prodrug, which is converted to its active form in vivo.

As used herein, the terms “treat,” “treating,” “treatment,” and the like refer to reducing or ameliorating a disease or condition, and/or symptoms associated therewith. It will be appreciated that, although not precluded, treating a disease or condition does not require that the disorder, condition or symptoms associated therewith be completely eliminated.

As used herein, the term “determining” refers to both quantitative and qualitative determinations and as such, the term “determining” is used interchangeably herein with “assaying,” “measuring,” and the like.

As used herein, the term “comprising”, “including” and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

As used herein, the term “mitochondrial dysfunction”, “mitochondrial disorder” or “mitochondrial disease” are used interchangeably and refers to a group of disorders that may occur when mitochondria fail to produce enough energy for the body to function properly. In another aspect, “mitochondrial disease” refers to a group of metabolic disorders. When the number or function of mitochondria in the cell are disrupted, less energy is produced and organ dysfunction results. Depending on which cells within the body have disrupted mitochondria or how many mitochondria are defective, the symptoms of mitochondrial disease can vary, such as, without limitation, fatigue, weakness, metabolic strokes, seizures, cardiomyopathy, arrhythmias, developmental or cognitive disabilities, diabetes mellitus, impairment of hearing, vision, growth, liver, gastrointestinal, or kidney function, and more. These symptoms can present at any age from infancy up until late adulthood including non-human.

As used herein, the term “metabolic disease” or “metabolic disorder” are used interchangeably and refers to a group of conditions that together raise risks of coronary heart disease, diabetes, stroke, and other serious health problems. They occur when the metabolism process fails and causes the body to have either too much or too little of the essential substances needed to stay healthy. The term “metabolic disease” used herein is associated with mitochondrial dysfunction.

As used herein, the term “cardiac disease” or “heart disease” are used interchangeably and refers to a group of disorders of the heart and blood vessels, such as, without limitation, coronary heart disease, cerebrovascular disease, peripheral arterial disease, rheumatic heart disease, congenital heart disease, and deep vein thrombosis and pulmonary embolism.

As used herein, the term “liver disease” refers to any of several conditions that can affect and damage liver, such as, without limitation, alcohol-related liver disease, non-alcoholic fatty liver disease, chronic hepatitis B and hepatocellular carcinoma. In one aspect, mitochondrial hepatopathies are a special group of liver diseases related to a problem with the way mitochondria are functioning. (Way S. Lee, M. D. et al., “Liver Disease in Mitochondrial Disorders”, Semin Liver Dis. 2007 August; 27 (3): 259-273)

As used herein, the term “diabetes” refers to a disease in which the body's ability to produce or respond to the hormone insulin is impaired, resulting in abnormal metabolism of carbohydrates and elevated levels of glucose in the blood and urine. In one aspect, mitochondrial diabetes is caused by poor functioning of insulin-producing cells in the pancreas and/or by the emergence of insulin resistance as part of a mitochondrial disorder.

As used herein, the term “obesity” refers to abnormal or excessive fat accumulation that presents a risk to health, partly attributable to excess food intake and physical inactivity. In certain aspect, obesity leads to an increase in the production of reactive oxygen species (ROS) and cause oxidative stress and in turn, can trigger mitochondrial dysfunction. In other aspects, mitochondrial dysfunction leads to a decrease in substrate oxidation, particularly fatty acids, resulting in lipid accumulation, including accumulation of ceramides and diacylglycerols. (Mora Murri et al., “MicroRNAs as regulators of mitochondrial dysfunction and obesity”, Am J Physiol Heart Circ Physiol 315: H291-H302, 2018)

As used herein, the term “aging” or “age-related change” are used interchangeably and refers to the result of an accumulation of damage to biomolecules due to the excessive production of highly toxic reactive oxygen species (ROS). In aged subjects, mitochondria are characterized by impaired function such as lowered oxidative capacity, reduced oxidative phosphorylation, decreased ATP production, significant increase in ROS generation, and diminished antioxidant defense. (Dimitry A. Chistiakov et al., “Mitochondrial Aging and Age-Related Dysfunction of Mitochondria”, Biomed Res Int. 2014; 2014:238463)

As used herein, the term “kidney disease” refers to the progression of acute kidney injury and chronic kidney disease. Acute kidney injury (AKI) is a sudden episode of kidney failure or kidney damage that happens within a few hours or a few days. AKI causes a build-up of waste products in blood and makes it hard for kidneys to keep the right balance of fluid in body. Chronic kidney disease, also called chronic kidney failure, involves a gradual loss of kidney function. Mitochondrial damages and dysfunction have been recognized as a leading factor to many chronic and acute renal diseases. (Divya Bhatia et al., “Mitochondrial dysfunction in kidney injury, inflammation, and disease: potential therapeutic approaches”, Kidney Res Clin Pract. 2020 Sep. 30; 39 (3): 244-258)

As used herein, the term “pancreas disorder” includes acute pancreatitis, chronic pancreatitis, hereditary pancreatitis, and pancreatic cancer. The pancreatitis occurs when digestive enzymes become activated while still in the pancreas, irritating the cells of the pancreas and causing inflammation. Acute pancreatitis is a sudden attack causing inflammation of the pancreas and is usually associated with severe upper abdominal pain. Chronic pancreatitis is the progressive disorder associated with the destruction of the pancreas. In some cases, pancreatitis is related to inherited abnormalities of the pancreas or intestine. In pancreatic β cells, mitochondria have been known to play a central role in coupling glucose metabolism to insulin exocytosis, thereby ensuring strict control of glucose-stimulated insulin secretion. Defects in mitochondrial function impair this metabolic coupling, and ultimately promote apoptosis and β cell death. (Abdoulaye Diane et al., “β-cell mitochondria in diabetes mellitus: a missing puzzle piece in the generation of hPSC-derived pancreatic β-cells?”, J Transl Med. 2022; 20:163)

As used herein, the term “cancer”, “tumor”, or “neoplasia” are used interchangeably and refer to a cell or population of cells whose growth, proliferation or survival is greater than growth, proliferation or survival of a normal counterpart cell, e.g., a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Cancers include benign, malignant, metastatic, and non-metastatic types, and include any stage (I, II, III, IV or V) or grade (G1, G2, G3, etc.). In particular, the term “cancer” includes carcinomas, such as squamous cell carcinoma, adenocarcinoma, adenosquamous carcinoma, anaplastic carcinoma, large cell carcinoma, and small cell carcinoma.

As used herein, the term “fatty acid oxidation disorders (FAODs)” or “Mitochondrial fatty acid oxidation disorders (FAODs)” are used interchangeably and refers to a group of inherited metabolic conditions that lead to an accumulation of fatty acids, and a decrease in cell energy metabolism. Each fatty acid oxidation disorder is associated with a specific enzyme defect in the fatty acid metabolic pathway and affects utilization of dietary and stored fat. Fatty acid oxidation disorders (FAODs) are inborn errors of metabolism due to disruption of either mitochondrial β-oxidation or the fatty acid transport using the carnitine transport pathway. In certain instances, the fatty acid oxidation disorders (FAODs) is, but not limited to, medium-chain acyl-CoA dehydrogenase deficiency (MCADD), very long-chain acyl-CoA dehydrogenase deficiency (VLCADD), long-chain 3-hydroxy acyl-CoA dehydrogenase deficiency (LCHADD) and trifunctional protein deficiency (TFPD), carnitine palmitoyltransferase type 1 deficiency (CPTID), carnitine-acylcarnitine translocase deficiency (CACTD), carnitine palmitoyltransferase type 2 deficiency (CPT2D), carnitine transporter deficiency (CTD), short-chain acyl-CoA dehydrogenase deficiency (SCADD), multiple acyl-CoA dehydrogenase deficiency (MADD), and 3-hydroxyacyl-CoA dehydrogenase deficiency (HADD). (J. Lawrence Merritt, I I et al., “Fatty acid oxidation disorders”, Ann Transl Med. 2018 December; 6 (24): 473)

As used herein, the term “effective amount” of a pharmaceutical compound identified by screening, as described in this disclosure, (e.g., a small molecule, a protein, a polypeptide, a peptide, a fusion protein, or an inhibitory nucleic acid (e.g., siRNA, miRNA, an antisense nucleic acid, or a peptide nucleic acid), an antibody, an antibody mimetic, or an aptamer) is an amount sufficient to improve mitochondrial function. An effective amount can be administered in one or more administrations, applications, or dosages.

As used herein, the term “therapeutically effective dose or amount” of a pharmaceutical compound identified by screening, as described in this disclosure, is intended an amount that, when administered as described herein, brings about a positive therapeutic response in treatment of mitochondrial diseases. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

As used herein, the term “pharmaceutically acceptable excipient or carrier” refers to an excipient that may optionally be included in the compositions of the invention and that causes no significant adverse toxicological effects to the patient.

As used herein, the term “pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

As used herein, the term “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

As used herein, the term “reference level” refers to an assay cutoff value that is used to assess diagnostic, prognostic, or therapeutic efficacy and that has been linked or is associated with herein various clinical parameters such as improvement of disease.

As used herein, the term “inhibitor” of mitochondrial dysfunction is a molecule that decreases or inhibits the activity associated with mitochondrial dysfunction when administered. The inhibitor may interact with mitochondria in target cells directly or may interact with another molecule that results in a decrease in the activity of mitochondrial dysfunction.

As used herein, the term “antibody” encompasses polyclonal antibodies, monoclonal antibodies as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); F(ab′)₂ and F (ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (scc, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, e.g., Riechmann et al. (1988) Nature 332:323-327; Verhocyan et al. (1988) Science 239:1534-1536; and U.K. Patent Publication No. GB 2,276, 169, published 21 Sep. 1994); and, any functional fragments obtained from such molecules, wherein such fragments retain specific-binding properties of the parent antibody molecule.

As used herein, the term “microRNA,” “miRNA,” and MiR” are interchangeable and refer to endogenous or artificial non-coding RNAs that are capable of regulating gene expression. It is believed that miRNAs function via RNA interference. When used herein in the context of inactivation, the use of the term microRNAs is intended to include also long non-coding RNAs, piRNAs, siRNAs, and the like. Endogenous (e.g., naturally occurring) miRNAs are typically expressed from RNA polymerase II promoters and are generated from a larger transcript.

As used herein, the term “siRNA” and “short interfering RNA” are interchangeable and refer to single-stranded or double-stranded RNA molecules that are capable of inducing RNA interference. SiRNA molecules typically have a duplex region that is between 18 and 30 base pairs in length.

As used herein, the term “piRNA” and “Piwi-interacting RNA” are interchangeable and refer to a class of small RNAs involved in gene silencing. piRNA molecules typically are between 26 and 31 nucleotides in length.

As used herein, the term “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against a target gene inducing mitochondrial dysfunction.

As used herein, the term “polynucleotide,” “oligonucleotide,” “nucleic acid” and “nucleic acid molecule” includes a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, microRNA, DNA: RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30:1911-1918; Elayadi et al. (2001) Curr. Opinion Invest. Drugs 2:558-561; Orum et al. (2001) Curr. Opinion Mol. Ther. 3:239-243; Koshkin et al. (1998) Tetrahedron 54:3607-3630; Obika et al. (1998) Tetrahedron Lett. 39:5401-5404.

As used herein, the term “CRISPR system” refers collectively to transcripts and other elements involved in the expression of or directing the activity of CRISPR-associated (“Cas”) genes. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence.

As used herein, the term “administering” a nucleic acid, such as an inhibitory or regulatory nucleic acid (e.g., microRNA, siRNA, piRNA, snRNA, antisense nucleic acid, or lncRNA), or a CRISPR system (expressing, e.g., a donor polynucleotide, guide RNA, Cas protein (e.g., Cas9, Cas12a, Cas12d, Cas13, or dCas9)) to a cell comprises transducing, transfecting, electroporating, translocating, fusing, phagocytosing, shooting or ballistic methods, etc., i.e., any means by which a nucleic acid can be transported across a cell membrane.

### II. Methods of Screening for a Pharmaceutical Compound for Improving Mitochondrial Function

The present disclosure provides methods for screening and identifying candidate agents that improve mitochondrial function in a subject. Specifically, the methods comprise a) contacting a biological sample obtained from a subject with a candidate agent; and b) measuring mitochondrial function or activity in the sample, wherein the presence of an elevated level of mitochondrial function or activity compared to that in a negative control that is not treated with the candidate agent indicates that the candidate agent improves mitochondrial function.

Methods of interest comprise obtaining a biological sample from a subject. The biological sample can vary and includes, e.g., tissues and cells. In some embodiments, the biological sample is, but not limited to, muscle tissue or cells, brain tissue or cells, nerves tissue or cells, liver tissue or cells, heart tissue or cells, kidney tissue or cells, pancreas tissue or cells, adipose tissue or cells, or gastrointestinal tract tissue or cells. In other embodiments, the biological sample may include a liquid sample, such as a blood plasma, urine, saliva, or cerebrospinal fluid (CSF). In some instances, the sample may be cell-free mitochondria. The biological sample can be obtained using any techniques known in the art. For example, biopsy testing may be performed to obtain the sample. In some embodiments, the biological sample may be prepared as isolated mitochondria or permeabilized cells or tissues using any techniques known in the art. For example, isolation of mitochondria through tissue or cell culture homogenization and differential centrifugation is routinely used for assessments of mitochondrial oxygen consumption, oxidant emission, and membrane potential in a variety of tissues. (Christopher G R Perry et al. “Methods for assessing mitochondrial function in diabetes”, Methodology Review, 2013 April; 62 (4): 1041-53). In the above methods, the biological sample refers to target tissue or target cells to measure mitochondrial function or activity.

In practicing the subject methods, the biological sample obtained from a subject is impaired tissues or cells associated with mitochondrial dysfunction. In some embodiments, the subject suffers from mitochondrial dysfunction or disease associated with mitochondrial dysfunction. In some instances, mitochondrial dysfunction or disease associated with mitochondrial dysfunction includes, but is not limited to, metabolic disease, cardiac disease, liver disease, diabetes, obesity, kidney disease, pancreas disorder, cancer, or fatty acid oxidation disorders (FAODs). In certain embodiments, the fatty acid oxidation disorders (FAODs) is the one selected from the group consisting of medium-chain acyl-CoA dehydrogenase deficiency (MCADD), very long-chain acyl-CoA dehydrogenase deficiency (VLCADD), long-chain 3-hydroxy acyl-CoA dehydrogenase deficiency (LCHADD) and trifunctional protein deficiency (TFPD), carnitine palmitoyltransferase type 1 deficiency (CPTID), carnitine-acylcarnitine translocase deficiency (CACTD), carnitine palmitoyltransferase type 2 deficiency (CPT2D), carnitine transporter deficiency (CTD), short-chain acyl-CoA dehydrogenase deficiency (SCADD), multiple acyl-CoA dehydrogenase deficiency (MADD), and 3-hydroxyacyl-CoA dehydrogenase deficiency (HADD). In certain instances, the cardiac disease also includes myocardial hypertrophy.

In some embodiments, the biological sample is muscle tissue or cells associated with mitochondrial dysfunction. For example, the muscle tissue may be, without limitation, skeletal muscle tissue, smooth muscle tissue, or cardiac muscle tissue. In other instances, the biological sample is brain tissue or cells associated with mitochondrial dysfunction. For example, the brain cells may be, but not limited to, neuronal, glial, or endothelial cells. In still other instances, the biological sample is heart tissue or cells associated with mitochondrial dysfunction. For example, the heart cells may be, but not limited to, cardiac fibroblasts (CFs), cardiomyocytes or endothelial cells (ECs). In yet other instances, the biological sample is kidney tissue or cells associated with mitochondrial dysfunction. For example, the kidney cells may be, but not limited to, kidney epithelial cells, or kidney endothelial cells. In further instances, the biological sample is pancreas tissue associated with mitochondrial dysfunction. For example, the pancreas cells may be, but not limited to, islet cells, acinar cells, ductal epithelial cells, or pancreatic beta cells. In still further instances, the biological sample is gastrointestinal tract tissue associated with mitochondrial dysfunction. For example, the gastrointestinal tract tissue may be, but not limited to, the mucosa, the submucosa, the muscularis propria, or the serosa.

In some embodiments, methods of interest alternatively comprise impairing mitochondrial function in the biological sample obtained from a subject who may not suffer from mitochondrial dysfunction or disease associated with mitochondrial dysfunction. Any techniques known in the art can be used to impair mitochondrial function in the sample. For example, pre-incubating the sample with palmitate can be used to prepare the sample having mitochondrial dysfunction.

In the above methods, the biological sample may be provided as an isolated tissues or cells. Any convenient format may be used for the assay, e.g. wells, plates, flasks, etc., preferably a high throughput format, such as multi-well plates. A candidate agent of interest is added to the reaction mixture with the target tissues or cells, for example in the presence of a candidate agent, and the effect of the candidate agent on mitochondrial activity is determined.

Methods of interest further include negative controls that is not treated with the candidate agent. Generally, a plurality of assay mixtures is run in parallel with different agent concentrations to obtain a differential response to the various concentrations. Typically, one of these concentrations serves as a negative control, i.e. at zero concentration or below the level of detection.

In the subject methods, the candidate agent is for improving mitochondrial function in target tissue or cells. In some embodiments, the candidate agent can be any molecule including, without limitation, a small molecule, a protein, a polypeptide, a peptide, a fusion protein, a nucleic acid, an oligonucleotide, a peptide nucleic acid, an antibody or fragment thereof, an antibody mimetic, an aptamer, or an inhibitory nucleic acid. In some instances, the candidate agent may reduce or inhibit activity inducing mitochondrial dysfunction. Inhibition may be complete or partial (i.e., all activity, some activity, or most activity is blocked by an inhibitor).

In practicing the subject methods, a variety of different candidate agents may be screened. In some embodiments, candidate agents encompass numerous chemical classes, e.g., small organic compounds having a molecular weight of more than 50 daltons and less than about 10,000 daltons, less than about 5,000 daltons, or less than about 2,500 daltons. In other embodiments, candidate agents can comprise functional groups necessary for structural interaction with proteins, e.g., hydrogen bonding, and can include at least an amine, carbonyl, hydroxyl or carboxyl group, or at least two of the functional chemical groups. In some embodiments, the candidate agents can comprise cyclical carbon or heterocyclic structures and/or aromatic or polyaromatic structures substituted with one or more of the above functional groups. In other embodiments, the candidate agents are also found among biomolecules including peptides, saccharides, fatty acids, steroids, purines, pyrimidines, derivatives, structural analogs or combinations thereof.

In embodiments, the candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. In some instances, the candidate agents are synthetic compounds. A number of techniques are available for the random and directed synthesis of a wide variety of organic compounds and biomolecules, including expression of randomized oligonucleotides. For example, WO 94/24314, hereby expressly incorporated by reference, which discusses methods for generating new compounds, including random chemistry methods as well as enzymatic methods. In other instances, the candidate agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the candidate agents are organic moieties. In this embodiment, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ccodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

In some embodiments, candidate agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit significant cytotoxic activity are considered candidate agents.

In some embodiments, the candidate agent is an antibody that specifically binds to and inhibits biological activity inducing mitochondrial dysfunction. Any type of antibody may be screened for the ability to inhibit activity inducing mitochondrial dysfunction by the methods described herein, including polyclonal antibodies, monoclonal antibodies, hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F (ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (scc, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, e.g., Riechmann et al. (1988) Nature 332:323-327; Verhocyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

In other embodiments, the candidate agent is an aptamer inhibiting biological activity inducing mitochondrial dysfunction. Aptamers may be isolated from a combinatorial library and improved by directed mutation or repeated rounds of mutagenesis and selection. For a description of methods of producing aptamers, see, e.g., Aptamers: Tools for Nanotherapy and Molecular Imaging (R. N. Veedu ed., Pan Stanford, 2016), Nucleic Acid and Peptide Aptamers: Methods and Protocols (Methods in Molecular Biology, G. Mayer ed., Humana Press, 2009), Aptamers Selected by Cell-SELEX for Theranostics (W. Tan, X. Fang eds., Springer, 2015), Cox et al. (2001) Bioorg. Med. Chem. 9 (10): 2525-2531; Cox et al. (2002) Nucleic Acids Res. 30 (20): e108, Kenan et al. (1999) Methods Mol. Biol. 118:217-231; Platella et al. (2016) Biochim. Biophys. Acta November 16 pii: S0304-4165 (16) 30447-0, and Lyu et al. (2016) Theranostics 6 (9): 1440-1452; herein incorporated by reference in their entireties.

In still other embodiments, the candidate agent is an antibody mimetic inhibiting biological activity inducing mitochondrial dysfunction. Any type of antibody mimetic may be used as an inhibitor, including, but not limited to, affibody molecules (Nygren (2008) FEBS J. 275 (11): 2668-2676), affilins (Ebersbach et al. (2007) J. Mol. Biol. 372 (1): 172-185), affimers (Johnson et al. (2012) Anal. Chem. 84 (15): 6553-6560), affitins (Krehenbrink et al. (2008) J. Mol. Biol. 383 (5): 1058-1068), alphabodies (Desmet et al. (2014) Nature Communications 5:5237), anticalins (Skerra (2008) FEBS J. 275 (11): 2677-2683), avimers (Silverman et al. (2005) Nat. Biotechnol. 23 (12): 1556-1561), darpins (Stumpp et al. (2008) Drug Discov. Today 13 (15-16): 695-701), fynomers (Grabulovski et al. (2007) J. Biol. Chem. 282 (5): 3196-3204), and monobodies (Koide et al. (2007) Methods Mol. Biol. 352:95-109).

In some embodiments, an inhibitor of target gene expression inducing mitochondrial dysfunction can include, but are not limited to, antisense oligonucleotides, inhibitory RNA molecules, such as miRNAs, siRNAs, piRNAs, and snRNAs, peptide nucleic acids, small molecule inhibitors, and CRISPR systems designed for genome, RNA transcript, or epigenome editing. Various types of inhibitors for inhibiting nucleic acid function are well known in the art. See e.g., International patent application WO/2012/018881; U.S. patent application 2011/0251261; U.S. Pat. No. 6,713,457; Kole et al. (2012) Nat. Rev. Drug Discov. 11 (2): 125-40; Sanghvi (2011) Curr. Protoc. Nucleic Acid Chem. Chapter 4: Unit 4.1.1-22; herein incorporated by reference in their entireties.

In another embodiment, the inhibitory RNA molecule having a single-stranded or double-stranded region that is at least partially complementary to the target sequence, e.g., about 75%, 80%, 85%, 90%, 91%, 92%, 93%, 94%, 95%, 96%, 97%, 98%, or 99% complementary to the target sequence. In other embodiments, the inhibitory RNA molecule may contain a region that has 100% complementarity to the target sequence. In certain embodiments, the inhibitory RNA molecule may be a double-stranded, small interfering RNA or a short hairpin RNA molecule (shRNA) comprising a stem-loop structure.

In some embodiments, inhibitors can be detectably labeled by well-known techniques. Detectable labels include, for example, radioactive isotopes, fluorescent labels, chemiluminescent labels, bioluminescent labels and enzyme labels. Such labeled inhibitors can be used to determine cellular uptake efficiency, quantitate binding of inhibitors at target sites, or visualize inhibitor localization.

In certain instances, the candidate agent is a dihydroceramide desaturase (Des) inhibitor. The dihydroceramide desaturase (Des) inhibitor is Des1 inhibitor, Des2 inhibitor or the combination thereof. Inhibition of dihydroceramide desaturase 1 (Des1) and/or Des2 activity in a human or animal subject has been known for the treatment of metabolic, cardiovascular, fibrotic, and autoimmune/chronic inflammatory diseases, as well as cystic fibrosis, various cancers and ischemia/reperfusion injury. Exemplary dihydroceramide desaturase (Des) inhibitors are described in U.S. Patent Publication No. 2020/0339535, the entire contents of which are incorporated by reference in their entirely.

In some embodiments, the candidate agent is added to the culture medium, and the culture medium is maintained under conventional conditions suitable for maintaining the target cells. Various commercially available systems have been developed for the growth of mammalian cells to provide for removal of adverse metabolic products, replenishment of nutrients, and maintenance of oxygen. By employing these systems, the medium may be maintained as a continuous medium, so that the concentrations of the various ingredients are maintained relatively constant or within a prescribed range.

A variety of other reagents may be included in the screening assay. These include reagents like salts, neutral proteins, e.g. albumin, detergents, etc., including agents that are used to facilitate optimal binding activity and/or reduce non-specific or background activity. Reagents that improve the efficiency of the assay, such as protease inhibitors, nuclease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity but may also be optimized to facilitate rapid high-throughput screening. In some embodiments, between 0.1 hour and 1 hour, between 1 hour and 2 hours, or between 2 hours and 4 hours, will be sufficient.

Methods of interest further comprise measuring the level of mitochondrial function or activity in the sample. Specifically, methods of interest comprises: (i) measuring the level of mitochondrial activity in the sample which is treated with the candidate agent; (ii) measuring the level of mitochondrial activity in negative control which is not treated with the candidate agent; (iii) comparing the levels of mitochondrial function or activity in the sample to that in the negative control; and (iv) determining whether the candidate agent improves mitochondrial function in the sample when the level of mitochondrial function or activity is higher than the level of that in the negative control. If the level of mitochondrial function or activity in the sample is not changed or not elevated compared to that in the negative control, the candidate agent is not effective to improve mitochondrial function in the sample. In some embodiments, methods of interest comprise isolating mitochondria from the sample in order to measure mitochondrial function or activity. The isolating mitochondria from the sample are performed using any technique known in the art. In other embodiments, the sample may be permeabilized cells or tissue.

In some embodiments, the mitochondrial function or activity in target tissue or cells is measured by oxygen consumption rate (OCR) and/or extracellular acidification rate (ECAR). Measurement of oxygen consumption rate (OCR) and/or extracellular acidification rate (ECAR) in target tissue or cells is used for assessment of the energy metabolism in target cells. The oxygen consumption rate (OCR) is a measurement of cellular respiration rate in target cells. The extracellular acidification rate (ECAR) is a measure of lactic acid levels, generated by anaerobic glycolysis. In the subject methods, the oxygen consumption rate (OCR) and extracellular acidification rate (ECAR) are measured before and after the addition of the candidate agent. The oxygen consumption rate (OCR) and extracellular acidification rate (ECAR) are measured by any techniques known in the art. For example, Seahorse XF Extracellular Flux Analyzer are utilized in order to measure oxygen consumption rate (OCR) and extracellular acidification rate (ECAR) in target cells.² (Birte Plitzko et al. “Measurement of Oxygen Consumption Rate (OCR) and Extracellular Acidification Rate (ECAR) in Culture Cells for Assessment of the Energy Metabolism”, Bio Protoc. 2018 May 20; 8 (10): e2850) In other embodiments, the mitochondrial function or activity in target tissue or cells is measured by mitochondrial calcium, superoxide, mitochondrial permeability transition, and membrane potential.

In some embodiments, a candidate agent identified as a pharmaceutical compound for improving mitochondrial function or an inhibitor of mitochondrial dysfunction in vitro cellular assays is further tested for its efficacy in treating disease associated with mitochondrial dysfunction in vivo, e.g., in an animal such as an animal model suffering from disease associated with mitochondrial dysfunction. For example, a candidate agent that improves mitochondrial function, identified as described herein, can be used in an animal model to determine the efficacy, toxicity, or side effects of treatment with such an agent. Alternatively, a candidate agent identified by the subject method can be used in an animal model to determine the mechanism of action of such an agent. Monitoring the efficacy of agents (e.g., drugs) on improving mitochondrial function can be applied not only in basic drug screening, but also in clinical trials. Furthermore, this disclosure pertains to uses of novel agents identified by the above-described methods for treatment of mitochondrial dysfunction.

In practicing the subject methods, a variety of assays may be used for this purpose, and in many embodiments, a candidate agent will be tested in different assays to confirm capability to improve mitochondrial function as well as efficacy in treating mitochondrial dysfunction. For example, biochemical assays may determine the ability of an agent to improve mitochondrial activity (e.g., enzymatic activity of the oxidative phosphorylation and/or metabolic activity). In addition, cell-based assays may be used, for example, for testing for oxygen consumption rate (OCR) or extracellular acidification rate (ECAR) of an impaired tissue associated with mitochondrial dysfunction in the absence or presence of a candidate agent.

### III. Pharmaceutical Compositions and Preparations Thereof

The candidate agents, identified by the screening method described Section II, can be formulated into pharmaceutical compositions optionally comprising one or more pharmaceutically acceptable excipients. The candidate agents, identified by the screening method described Section II are meant to interchangeably “pharmaceutical compounds” in this disclosure. In some embodiments, exemplary excipients include, without limitation, carbohydrates, inorganic salts, antimicrobial agents, antioxidants, surfactants, buffers, acids, bases, and combinations thereof. In other embodiments, Excipients suitable for injectable compositions include water, alcohols, polyols, glycerine, vegetable oils, phospholipids, and surfactants. In some instances, a carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

In therapeutic applications, the pharmaceutical compositions of interest can be formulated for a variety of modes of administration, including systemic and topical or localized administration. Techniques and formulations generally may be found in Remington: The Science and Practice of Pharmacy (20th ed.) Lippincott, Williams & Wilkins (2000). Depending on the route of administration, the pharmaceutical compositions of interest can be coated with or disposed in a selected material to protect it from natural conditions which may detrimentally affect its ability to perform its intended function.

In some embodiments, the pharmaceutical composition of interest can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

In other embodiments, an antioxidant can be present in the composition as well. Antioxidants are used to prevent oxidation, thereby preventing the deterioration of the pharmaceutical components identified by screening methods as described Section II. For example, suitable antioxidants for use in the present disclosure include ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

In still other embodiments, acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the pharmaceutical component identified by the screening method described Section II (e.g., when contained in a drug delivery system) in the pharmaceutical composition will vary depending on a number of factors but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

In some embodiments, the amount of any individual excipient in the pharmaceutical composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000.

In other embodiments, the pharmaceutical compositions of interest encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

In some embodiments, the pharmaceutical preparations herein can also be housed in a syringe, an implantation device, or the like, depending upon the intended mode of delivery and use. Preferably, the compositions comprising one or more pharmaceutical component identified by the screening method described Section II, or nucleic acids encoding them, are in unit dosage form, meaning an amount of a conjugate or composition of the invention appropriate for a single dose, in a premeasured or pre-packaged form.

In other embodiments, the pharmaceutical compositions of interest may optionally include one or more additional agents, such as one or more other drugs for treating mitochondrial dysfunction or other medications.

### IV. Methods of Treating Mitochondrial Dysfunction

The present disclosure also provides methods of treating mitochondrial dysfunction or disease associated with mitochondrial dysfunction in a subject in need of treatment thereof. Specifically, the method comprises administering to the subject an effective amount of a pharmaceutical compound identified by the screening method described Section II. In practicing the subject methods, the pharmaceutical compound identified by the screening method described Section II can be used alone or in combination with one or more additional therapeutic agents in admixture with a pharmaceutically acceptable excipient as described Section III.

In the above methods, the mitochondrial dysfunction or disease associated with mitochondrial dysfunction includes, but is not limited to, metabolic disease, cardiac disease, liver disease, diabetes, obesity, kidney disease, pancreas disorder, cancer, or fatty acid oxidation disorders (FAODs). In certain embodiments, the fatty acid oxidation disorders (FAODs) is the one selected from the group consisting of medium-chain acyl-CoA dehydrogenase deficiency (MCADD), very long-chain acyl-CoA dehydrogenase deficiency (VLCADD), long-chain 3-hydroxy acyl-CoA dehydrogenase deficiency (LCHADD) and trifunctional protein deficiency (TFPD), carnitine palmitoyltransferase type 1 deficiency (CPTID), carnitine-acylcarnitine translocase deficiency (CACTD), carnitine palmitoyltransferase type 2 deficiency (CPT2D), carnitine transporter deficiency (CTD), short-chain acyl-CoA dehydrogenase deficiency (SCADD), multiple acyl-CoA dehydrogenase deficiency (MADD), and 3-hydroxyacyl-CoA dehydrogenase deficiency (HADD).

In the subject methods, the pharmaceutical compounds of interest are administered to the subject having mitochondrial dysfunction. Examples of routes of administration which can be used include injection (subcutaneous, intravenous, parenterally, intraperitoneally, intrathecal, etc.), oral, inhalation, and transdermal. The injection can be bolus injections or can be continuous infusion. In some embodiments, the component of interest may be administered alone, co-administered with or in conjunction with a pharmaceutically acceptable carrier. The compound also may be administered as a prodrug, which is converted to its active form in vivo.

In some embodiments, the pharmaceutical compounds of interest are effective over a side dosage range. In some instances, in the treatment of adult humans, dosages from 0.01 to 1000 mg, from 0.5 to 100 mg, from 1 to 50 mg, from 1-30 mg, from 1 to 20 mg, from 5 to 20 mg, from 10 to 20 mg, from 10 to 19 mg, from 10 to 18 mg, from 10 to 17 mg or from 10 to 16 mg per day may be used. The exact dosage will depend upon the route of administration, the form in which the compound is administered, the subject to be treated, the body weight of the subject to be treated, and the preference and experience of the attending physician.

In some embodiments, methods of interest further comprise obtaining the level of mitochondrial function or activity in a biological sample from the subject, which is treated with the pharmaceutical compound identified by the screening method described Section II. And then, the level of mitochondrial activity is compared to a reference level. The reference level refers to an assay cutoff value that is used to assess therapeutic efficacy and corresponds to the level of a negative control described in Section II. If the level of mitochondrial function or activity in the sample is elevated compared to the reference level, the pharmaceutical compound would be considered to provide therapeutic efficacy. If the level of mitochondrial function or activity in the sample is not changed or not elevated compared to the reference level, the pharmaceutical compound would not be considered to provide therapeutic efficacy.

In embodiments, methods of interest result in a decrease in the severity of mitochondrial dysfunction in a subject. The term “decrease” is meant to inhibit, suppress, attenuate, diminish, arrest, or stabilize a symptom of a disease or condition. In some embodiments, the pharmaceutical compounds of interest would be considered as an inhibitor of mitochondrial dysfunction that decreases or inhibits the activity associated with mitochondrial dysfunction. The inhibitor may interact with mitochondria in target cells directly or may interact with another molecule that results in a decrease in the activity of mitochondrial dysfunction.

### V. Assaying Kits for Screening a Pharmaceutical Compound for Improving Mitochondrial Function

The present disclosure further provides assaying kits for screening a pharmaceutical compound for improving mitochondrial function. More specifically, kits of the present disclosure comprise an impaired tissue associated with mitochondrial dysfunction and a candidate agent. The impaired tissue associated with mitochondrial dysfunction and the candidate agent are described in Section II of the present disclosure.

### VI. Examples

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in protein structure without affecting the biological action in kind or amount. All such modifications are intended to be included within the scope of the appended claims.

### Example 1

Various in vitro cellular assays of mitochondrial function are available for testing compounds and drug candidates. By way of example, and in the case of kidney function, human kidney 2 (HK-2) cells can be assayed for mitochondrial activity, including oxygen consumption rate (OCR) and extracellular acidification rate (ECAR).

### Procedure:

HK-2 is a proximal tubular cell line derived from normal human kidney. Two days prior to assay, HK-2 cells are seeded into a 96-well Seahorse Plate at 12× 10³ cells/well. One day prior to assay, the seeded HK-2 cells are pre-incubated with vehicle, a representative DES1 inhibitor, palmitate, or palmitate+a representative DES1 inhibitor. On the day of the mitochondrial assay, serial injections of oligomycin, FCCP, and rotenone/antimycin A are used in order to assess function at key mitochondrial complexes.

### Result:

It is anticipated that palmitate pre-incubation will impair OCR, and that this will be prevented by treatment with a representative DES1 inhibitor. This result demonstrates that a representative DES1 inhibitor protects and/or improves mitochondrial function.

### Example 2

Various in vitro cellular assays of mitochondrial function are available for testing compounds and drug candidates. By way of example, and in the case of heart function, H9C2 rat cardiomyoblast cells treated with palmitate can be assayed for mitochondrial activity, including oxygen consumption rate (OCR) and extracellular acidification rate (ECAR). Moreover, these assays could be performed in cultured cardiomyocytes from humans with fatty acid oxidation disorders (in the absence of palmitate).

### Procedure:

H9C2 cells of embryonic rat cardiomyocytes are a subclonal line of the original clonal cell line derived from embryonic BDIX rat heart tissue. Two days prior to assay, H9C2 cells are seeded into a 96-well Seahorse Plate at 12×10³ cells/well. One day prior to assay, the seeded HK-2 cells are pre-incubated with vehicle, a representative DES1 inhibitor, palmitate, or palmitate+a representative DES1 inhibitor. On the day of the mitochondrial assay, serial injections of oligomycin, FCCP, and rotenone/antimycin A are used in order to assess function at key mitochondrial complexes. Additional readouts include determining the expression of various markers of hypertrophy (including Myh7, Nppa, Nppb, and Tnnc1), fibrosis (including TGFβ1, Fn1, Col1a1, Col6a3, and Col3a1), inflammation (including IL-6, MCP1, TNFα, IL-1), apoptosis (including BCL2), and mitochondrial biogenesis (including Tfam).

### Result:

It is anticipated that palmitate pre-incubation will impair OCR, and that this will be prevented by treatment with a representative DES1 inhibitor. Moreover, it is anticipated that OCR will be basally impaired in cardiomyocytes from humans with a fatty acid oxidation disorder, and that this will be prevented and/or resolved by treatment with a representative DES1 inhibitor. It is also anticipated that the expression levels of various markers of hypertrophy, fibrosis, inflammation, apoptosis, and mitochondrial biogenesis will be abnormal in rat cardiomyocytes treated with palmitate as well as cardiomyocytes from humans with a fatty acid oxidation disorder, and that these changes will be prevented and/resolved by treatment with a representative DES1 inhibitor. This result demonstrates that a representative DES1 inhibitor protects and/or improves mitochondrial function.

### Example 3

There are various animal models of fatty acid oxidation disorders (FAODs) for assessing drug efficacy. By way of example, homozygous genetic ablation of Acadl in mice causes a phenotype resembling the metabolic and cardiac manifestations of very long-chain acyl-CoA dehydrogenase deficiency (VLCADD), a rare genetic disorder of fatty acid metabolism that is transmitted in an autosomal recessive pattern. Acadl^{-/-} mice exhibit elevations in C14:1, C16, and C18:1 acylcarnitines, myocardial hypertrophy, decreased ejection fraction, decreased stroke volume, decreased heart rate, and decreased cardiac output.

### Result:

Treatment of these mice with a representative DES1 inhibitor is anticipated to reverse the cardiac phenotype, including reduction in myocardial hypertrophy (as determined by a reduction in the heart weight/tibial length) and improvement in cardiac function (as determined by echocardiographic/tissue doppler assessment of ejection fraction and fractional shortening). This result demonstrates that a representative DES1 inhibitor can improve cardiac function and quality of life in patients with various fatty acid oxidation disorders.

### Rationale for Human Efficacious Dose Level of 10-16 Milligrams Per Day:

It was estimated that a CNT2130 dose level of 10-16 mg/day will confer disease-modifying efficacy in humans. In rats, a CNT2130 dose level of 1 mg/kg is sufficient to inhibit DES1 and lower plasma ceramides by at least 50% compared to vehicle control. In mice, a 50% reduction in plasma ceramides is associated with robust disease-modifying efficacy in models of insulin resistance, liver, kidney, and heart diseases. When adjusted for body surface area, the human dose level equivalent to the rat dose level of 1 mg/kg is 0.16 mg/kg. which translates into a range of 10-16 mg/day (assuming human body weights of 60-100 kg).

What is claimed is:

1. A method of screening for a pharmaceutical composition for improving mitochondrial function, the method comprising:
a) contacting a biological sample obtained from a subject with a candidate agent; and
b) measuring mitochondrial function in the sample, wherein the presence of an elevated level of mitochondrial function compared to that in a negative control sample that is not treated with the candidate agent indicates that the candidate agent improves mitochondrial function.

2. The method of claim 1, wherein the biological sample is an impaired tissue associated with mitochondrial dysfunction.

3. The method of claim 1, wherein the biological sample is muscle tissue, brain tissue, liver tissue, heart tissue, kidney tissue, pancreas, adipose tissue, or gastrointestinal tract tissue.

4. The method of claim 1, wherein the biological sample is human kidney 2 (HK-2) cells.

5. The method of claim 1, wherein the biological sample is H9C2 rat cardiomyoblast cells or cultured cardiomyocytes from a human with a fatty acid oxidation disorder.

6. The method of claim 1, wherein the subject is having or suspected to have mitochondrial dysfunction.

7. The method of claim 6, wherein the mitochondrial dysfunction includes metabolic disease, skeletal muscle disease, cardiac disease, liver disease, diabetes, obesity, kidney disease, pancreas disorder, cancer, or fatty acid oxidation disorders (FAODs).

8. The method of claim 1, wherein the candidate agent improves mitochondrial function.

9. The method of claim 1, wherein the candidate agent is a small molecule, a peptide, a protein, an enzyme, an antibody, an antibody mimetic, an aptamer, or an inhibitory nucleic acid.

10. The method of claim 1, wherein the candidate agent is a dihydroceramide desaturase (Des) inhibitor.

11. The method of claim 10, wherein the dihydroceramide desaturase (Des) inhibitor is Des1 inhibitor, Des2 inhibitor or the combination thereof.

12. The method of claim 1, wherein the mitochondrial function in the sample is measured by oxygen consumption rate (OCR) and/or extracellular acidification rate (ECAR).

13. A method of treating mitochondrial dysfunction in a subject in need of treatment thereof, comprising the step of administering to the subject an effective amount of a pharmaceutical compound identified by the method of claim 1.

14. The method of claim 13, further comprising: obtaining the level of mitochondrial activity in a biological sample from the subject, wherein the presence of an elevated level of mitochondrial function compared to a reference level indicates efficacy of the pharmaceutical compound.

15. The method of claim 13, wherein the mitochondrial dysfunction includes metabolic disease, skeletal muscle disease, cardiac disease, liver disease, diabetes, obesity, kidney disease, pancreas disorder, cancer, or fatty acid oxidation disorders (FAODs).

16. The method of claim 15, wherein the fatty acid oxidation disorders (FAODs) is the one selected from the group consisting of medium-chain acyl-CoA dehydrogenase deficiency (MCADD), very long-chain acyl-CoA dehydrogenase deficiency (VLCADD), long-chain 3-hydroxy acyl-CoA dehydrogenase deficiency (LCHADD) and trifunctional protein deficiency (TFPD), carnitine palmitoyltransferase type 1 deficiency (CPT1D), carnitine-acylcarnitine translocase deficiency (CACTD), carnitine palmitoyltransferase type 2 deficiency (CPT2D), carnitine transporter deficiency (CTD), short-chain acyl-CoA dehydrogenase deficiency (SCADD), multiple acyl-CoA dehydrogenase deficiency (MADD), and 3-hydroxyacyl-CoA dehydrogenase deficiency (HADD).

17. An assaying kit for screening a pharmaceutical compound for improving mitochondrial function, the kit comprising an impaired tissue associated with mitochondrial dysfunction and a candidate agent.

18. The assaying kit of claim 17, wherein the candidate agent is dihydroceramide desaturase (Des) inhibitor.

19. The assaying kit of claim 18, wherein the dihydroceramide desaturase (Des) inhibitor is Des1 inhibitor, Des2 inhibitor or the combination thereof.

20. The assaying kit of claim 17, wherein the impaired tissue associated with mitochondrial dysfunction is obtained from muscle tissue, brain tissue, liver tissue, heart tissue, kidney tissue, pancreas, adipose tissue, or gastrointestinal tract tissue.
